(19) United States
(12) Patent Application Publication
(10) Pub. No.: US 2007/0077294 A1
(43) Pub. Date: Apr. 5, 2007

(54) Capsules comprising topiramate

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/540,688

(22) Filed: Oct. 2, 2006

(30) Foreign Application Priority Data
Oct. 4, 2005 (CA) 2,521,272

(51) Int. Cl. A61K 9/48; A61K 31/7008

(52) U.S. Cl. 424/451; 514/23

(57) ABSTRACT

Pharmaceutical capsules containing uncoated particles comprising topiramate and an acid-insoluble polymer.

### BACKGROUND

Topiramate is a compound that has anticonvulsant activity. It is sold in the United States and elsewhere under the tradename Topamax™ in tablets in strengths of 25, 50, 100 and 200 mg.

It is desirable to have anticonvulsant products available in a form other than tablets that can be used by children and other patients who have difficulty in swallowing tablets.

For topiramate, the development of an alternate form for that purpose was found difficult because topiramate has an extremely bitter taste.

The problem has been overcome by a new topiramate composition now available in the United States and elsewhere under the tradename Topamax™ Sprinkle Capsules. This composition is in the form of 2-piece hard gelatin capsules that contain small coated particles. The coated particles are comprised of core particles that contain topiramate, which are coated with a taste-masking coating. The patient uses a capsule by opening it, sprinkling the coated particles onto soft food, such as apple sauce, and ingesting the coated particles along with the food, without chewing, so as to avoid breaking the taste-masking coating on the particles.

This composition is the subject of pending U.S. patent application Ser. No. 10/748,764.

While this composition overcomes the problem caused by the very bitter taste of topiramate, the process of making the coated particles is relatively expensive. The objective of the present invention is thus to provide a simpler alternative.

™—Registered Trademark.

### DESCRIPTION OF THE INVENTION

It has been found that the bitter taste of topiramate can be overcome by using particles that are uncoated (i.e. have no taste-masking coating), provided that the particles comprise, along with the topiramate, a polymer that is insoluble in acidic aqueous media. The polymer serves to retard dissolution of the particles in saliva.

Compositions of the present invention are thus capsules containing particles that are uncoated and comprise topiramate and one or more acid-insoluble polymers, wherein the amount of acid-insoluble polymer is at least double the amount of topiramate by weight.

The acid-insoluble polymer may be insoluble in water at all pH's, such as, for example, ethylcellulose or cellulose acetate.

Alternatively, it may be a polymer that is insoluble in water at acidic pH (i.e. pH below about 5), but soluble at basic pH's such as, for example, polyvinyl acetate phthalate.

Alternatively, a mixture of any two or more such polymers may be used.

The ratio of acid-insoluble polymer to topiramate by weight will preferably be from 2 to about 10, and more preferably from 3 to about 6.

Preferably the size distribution of the particles will be such that most of the mass of the particles is in particles of size between 0.5 mm and 2.5 mm.

### EXAMPLE 1

Topiramate was mixed with polyvinyl acetate phthalate and ethylcellulose powders in the ratio of 3 parts polyvinyl acetate phthalate and 1 part ethylcellulose to 1 part topiramate by weight. The powder mixture was compacted, and the compacted material was milled through a #12 screen (12 wires per inch). The resulting particles were then sifted on a #20 screen (20 wires per inch). The particles that went through the #20 screen were discarded. The particle retained on the #20 screen had a size range of from about 1 mm to about 2 mm.

These particles were filled into size 3 capsules at a net fill weight of 75 mg. As the mixture contained 1 part topiramate per 5 parts total, each capsule contained about 15 mg of topiramate.

The content of one of these capsules was sprinkled onto apple sauce. It was found that, when the mixture in apple sauce was ingested, the taste was not significantly more bitter than when content of a 15 mg capsule of Topamax™ was similarly sprinkled onto apple sauce and ingested.

1. A pharmaceutical capsule containing uncoated particles comprising topiramate and an acid-insoluble polymer, wherein the amount of acid-insoluble polymer is at least twice the amount of topiramate by weight.

2. A capsule of claim 1, wherein the polymer comprises a polymer that is insoluble in water at all pH's.

3. A capsule of claim 1, wherein the polymer comprises ethylcellulose.

4. A capsule of claim 1, wherein the polymer comprises cellulose acetate.

5. A capsule of claim 1 wherein the polymer comprises a polymer that is insoluble in water at pH below about 5 but is soluble in water at basic pH.

6. A capsule of claim 5 wherein the polymer comprises polyvinyl acetate phthalate.

7. A capsule of claim 1, wherein the ratio of polymer to topiramate is from 2 to about 10.

8. A capsule of claim 1, wherein the ratio of polymer to topiramate is from 3 to about 6.

9. A capsule of claim 1, wherein the size distribution of the particles is such that most of the mass of the particles is in particles of size between 0.5 mm and 2.5 mm.
